(19) United States
(12) Plant Patent Application Publication
(10) Pub. No.: US 2003/0024025 P1
(43) Pub. Date: Jan. 30, 2003

(54) Chrysanthemum named 'Dark Reagan Mundo'

(76) Inventor: Robert Noodelijk, Woubrugge (NL)

Correspondence Address: PARKHURST & WENDEL, 1421 PRINCE STREET, SUITE 210, ALEXANDRIA, VA, 22314, US

(21) Appl. No.: 09/906,688

(22) Filed: Jul. 18, 2001

(51) Int. Cl. A01H 5/00

(52) U.S. Cl. PLT/297

(57) ABSTRACT

A Chrysanthemum plant named ‘Dark Reagan Mundo’ characterized by its medium sized blooms with white ray-florets and yellow-green disc florets.

### RELATED APPLICATIONS

‘Dark Reagan Mundo’ is related to ‘Pearl Reagan Mundo’ (unpatented) and the co-pending ‘Yellow Reagan Mundo’ (#______) ‘Dark Reagan Mundo’ and ‘Yellow Reagan Mundo’ are both a natural sport of ‘Pearl Reagan Mundo’.

### BACKGROUND OF THE INVENTION

‘Dark Reagan Mundo’ is a product of a beding and selection program which had the objective of finding color mutants of ‘Pearl Reagan Mundo’. The new plant of the present invention comprises a new and distinct cultivar of chrysanthemum plant that is a natural occuring sport of a parent chrysanthemum named ‘Pearl Reagan Mundo’. A comparison with Parent chrysanthemum ‘Pearl Reagan Mundo’ is also made in this application. The new cultivar was discovered as a whole plant mutation in September 1996 by Rob Noodelijk in a controlled environment (greenhouse) in Rijsenhout Holland. The first act of asexual reproduction of ‘Dark Reagan Mundo’ was accomplished when vegetative cuttings were taken from the initial selection in November 1996 in Rijsenhout Holland.

### SUMMARY OF THE INVENTION

The present invention is a new and distinct variety of chrysanthemum bearing medium sized blooms with white ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a stem of the cultivar in full bloom.

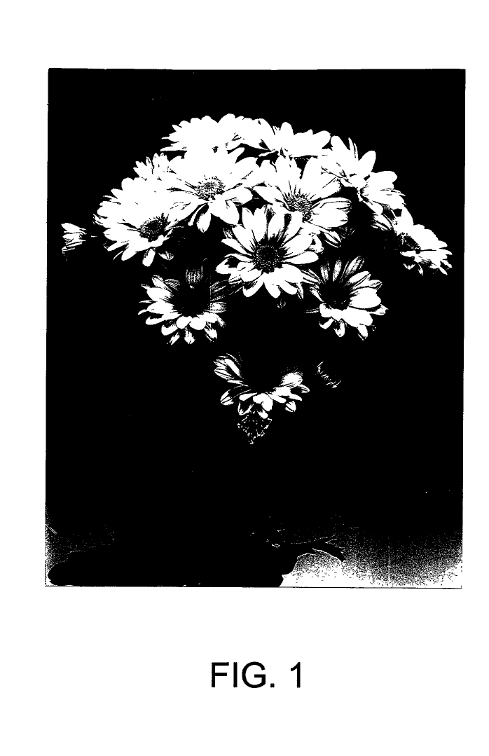

### DESCRIPTION OF THE INVENTION

This new variety of chrysanthemum is of the botanical classification dendranthema Grandiflora. The observations and measurements were gathered from plants grown in April/May a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 50 days. After the long day period to flowering growth retardants were applied 2 to 3 times in an average dose of 2.5 gram/liter water. No tests were done on disease or insect resistance or susceptibility. This new variety produces medium sized blooms with white ray-florets and yellow-green disc-florets blooming on the plant for 28 days. This new variety of chrysanthemum has been found to retain its distinctive characteristics throughout successive propagations however the phenotype may vary significantly with variations in environment such as light intensity and temperature. To show the phenotype as described ‘Dark Reagan Mundo’ may be planted without assimilation lightning between week 50 and 40 under greenhouse conditions in Holland. With assimilation lightning (minimum level 2500 lux) it may be planted year round under greenhouse conditions in Holland.

From the cultivars known to inventor the most similar existing cultivars in comparison to ‘Dark Reagan Mundo’ are ‘Pearl Reagan Mundo’, ‘Dark Reagan’ and ‘Blue Pink Elite Reagan’.

When ‘Dark Reagan Mundo’, ‘Pearl Reagan Mundo’ , ‘Dark Reagan’ and ‘Blue Pink Elite Reagan’ are being compared the following differences and similarities are noticed. (A) The difference of ‘Dark Reagan Mundo’ and ‘Pearl Reagan Mundo’ is the flower color for which characteristic ‘Dark Reagan Mundo’ has been selected out of ‘Pearl Reagan Mundo’. All other characteristics of ‘Dark Reagan Mundo’ and ‘Pearl Reagan Mundo’ are the same. (B) The differences of ‘Dark Reagan Mundo’ and ‘Dark Reagan’ are (1) flower form (2) flower size (3) spray formation and flower count and (4) tolerance for a lower night temperature. All other characteristics of ‘Pearl Reagan Mundo’ and ‘Reagan’ are similar. (C) The differences of ‘Dark Reagan Mundo’ and ‘Blue Pink Elite Reagan’ are (1) size and color of the flower disc (2) growth rate (3) spray formation (4) tolerance for a lower night temperature. All other characteristics of ‘Dark Reagan Mundo’ and ‘Blue Pink Elite Reagan’ are similar.

(A) Flower color: ‘Dark Reagan Mundo’ has pink ray-florets instead of pale pink ray-florets. (B) (1) Flower form: The ray-florets of ‘Pearl Reagan Mundo’ are less broad and more pointed and they are more upright. The flower-disc is smaller, greener and more regular. (2) Flower size: The flower is a little smaller. (3) The spray formation: The spray formation is more flat, less pyramidal, mostly a little wider and the flower count is higher. (4) Temperature tolerance: ‘Pearl Reagan Mundo’ and all its color mutants can be grown at a lower night temperature. The required minimum temperature for ‘Reagan’ (under all circumstances) is 17° C. Under high light conditions ‘Pearl Reagan Mundo’ can be grown at 14° C. This has been tested and confirmed in poor heated crops under high light conditions flowering in February 1997 and 1998 in Italy and Spain. (C) ‘Dark Reagan Mundo’ has almost the same flower shape as ‘Blue Pink Elite Reagan’ . The flower disc however is not so small. The growth is less vigorous instead of more vigorous. The spray formation is more spreading, with a bigger angle of the peduncles with the stem instead of more upright for ‘Blue Pink Elite Reagan’ and the most essential difference: ‘Blue Pink Elite Reagan’ does not have a low temperature tolerance as ‘Dark Reagan Mundo’.

The following is a description of the plant and characteristics that distinguish ‘Dark Reagan Mundo’ as a new and distinct variety.

The color designations are taken from the plant itself. Accordingly, any discrepancies between the color designations and the colors depicted in the photographs are due to photographic tolerances. The color chart used in this description is: The Royal Horticultural Society color chart, edition 1995.

### Botanical Description of CULTIVAR ‘Dark Reagan Mundo’

Bud:

Size.—Medium Cross-section+/−1.0 cm height+/−0.7 cm.

Outside color.—Greyed-purple 186 A.

Involucral bracts.—2 rows, length 7 mm, width 3 mm.

Involucral bracts among disc-florets.—Not present.

Involucral bracts color.—Green 138 B.

Bloom:

Type.—Single.

Height.—Flat.

Size.—Medium.

Fully expanded.—6-6.5 cm.

Number of blooms per single stem.—Average of 12-14.

Seeds.—Not produced.

Performance (blooming period).—28 days.

Color:

Center of the flower (disc-florets).—Immature Yellow-green 144 A in the center to yellow 12 A along the border, mature Yellow 12 A.

Color of upper surface of the majority of the ray-florets.—Purple 75 C.

Color of the lower surface of the majority of the ray-florets.—Purple 75 D.

Tonality from distance.—A pink daisy with a yellow-green disc.

Discoloration to color.—Purple 75 D.

Ray florets:

Texture.—Upper and under side smooth.

Number.—26-30 (two rows).

Cross-section.—Convex.

Longitudinal axis of majority.—Straight to slightly reflexing.

Length of corolla tube.—Very short.

Ray-floret length.—3.5 cm.

Ray-floret width.—1.2 cm.

Ratio length/width.—Medium.

Shape of tip.—Pointed, sometimes dentate.

Disc florets:

Disc diameter (when visible).—Medium (1.3-1.5 cm).

Distribution of disc florets.—Numerous, visible at all stages of flowering.

Type.—Tubular.

Color.—Immature Yellow-green 144 A mature yellow 12 A.

Receptacle shape.—Conical raised.

Reproductive Organs:

Stamen (present in disc florets only).—Yellow, thick, 3 mm in length.

Stamen color.—Yellow 12 B.

Pollen.—Present.

Styles (present in both ray and disc florets).—Green, thick.

Style color.—Green 143 B.

Style length.—+/−5 mm.

Stigmas.—Yellow.

Stigma width.—+/−1 mm.

Ovaries.—Enclosed in calyx.

Plant:

Form.—A spray mum meant for erect culture.

Growth habit.—Erect.

Growth rate.—Medium.

Height.—100-115 cm.

Internodes.—2.5-3.0 cm.

Spray formation.—Corymbiform (funnel-shaped with a flat umbel on top).

Peduncle length.—Near the top +/−12 cm, near the middle +/−16 cm, near the bottom +/−21 cm.

Peduncle color.—Green 138 B.

Peduncle, attachment.—Medium ,not weak not strong.

Peduncle, angle with stem.—Medium to large, 40°.

Stem color.—Green 138 B.

Stem strength.—Medium to strong.

Stem brittleness.—Absent.

Stem anthocyanin coloration.—Present.

Flowering response (photo-response (photo-periodic controlled crop, not natural season).—50 days.

Foliage:

Color.—Upper side green 137 A. Under side green 137 C

Size.—length +/−12 cm, width +/9 cm.

Quantity (i number per single stem).—26-32.

Shape.—Oval.

Texture upper side.—Scabrous.

Texture under side.—Pubescent.

Venation arrangement.—Palmate.

Shape of the margin.—Lobed.

Shape of base of sinus between lateral lobes.—Round.

Margin of sinus between lateral lobes.—Converging sometimes asymmetric.

Shape of base.—Rounded.

Apex.—Mucronate.

TABLE 2
Differences with comparison varieties

| | | | | ‘Blue Pink |
|---|---|---|---|---|
| | ‘Dark Reagan | ‘Pearl Reagan | ‘Dark | Elite |
| | Mundo’ | Mundo’ | Reagan’ | Reagan’ |
| Color | Purple 75 C | Purple 75 D | Red-purple | Purple 77 D |
| of Ray | | | 69 B | |
| florets | | | | |
| Flower | 1.3-1.5 cm | 1.3-1.5 cm | 1.5 cm | 1.0-1.2 cm |
| disc size | | | | |
| Growth | Medium | Medium | Medium | Vigorous |
| rate | | | | |
| Height | 100-115 cm | 100-115 cm | 100-125 cm | 105-130 cm |
| Peduncle, | 40° | 40° | 30°-35° | 30° |
| angle with | | | | |
| stem | | | | |
| Required | 14° C. | 14° C. | 17° C. | 17° C. |
| minimum | | | | |
| night | | | | |
| temper- | | | | |
| ature | | | | |

I claim:

1. A new and distinct variety of chrysanthemum plant as described and illustrated.
